(19) United States
(12) Patent Application Publication
(10) Pub. No.: US 2024/0180686 A1
(43) Pub. Date: Jun. 6, 2024

(54) SYSTEM, APPARATUSES, DEVICES, AND METHODS FOR AN ANALYTE DIFFUSIVE IMPLANTABLE DEVICE

(71) Applicant: Neurotech USA, Inc., Cumberland, RI (US)

(72) Inventors: Konrad A. KAUPER, Sutton, MA (US); John F. MILLS, Wakefield, RI (US); Sandy SHERMAN, Fairhaven, MA (US); Arne M. NYSTUEN, Brookline, MA (US)

(21) Appl. No.: 17/773,161

(22) PCT Filed: Nov. 2, 2020
(86) PCT No.: PCT/US2020/058589

§ 371 Date: Apr. 29, 2022

Related U.S. Application Data
(60) Provisional application No. 62/929,619, filed on Nov. 1, 2019.
(60) Provisional application No. 62/930,504, filed on Nov. 4, 2019.

(51) Int. Cl. A61F 2/02; A61F 2/00; A61F 9/00

(52) U.S. Cl. CPC A61F 2/022; A61F 2/0095; A61F 9/0017; A61F 2220/0008; A61F 2250/0068

(57) ABSTRACT

Embodiments of the present disclosure include, for example, a medical device system is provided and includes an analyte diffusive implantable device (ADID), which comprises a cavity, a cell-scaffolding material (CSM) arranged within the cavity and configured to hold or otherwise retain a plurality of living cells engineered to secrete a therapeutic protein-based drug, and a porous hollow fiber membrane at least partially enclosing the cavity, where at least a portion of the membrane is adhered to the CSM by a high durometer adhesive which creates a hemispheric seal at both ends of the membrane, and a filling port having a docking means at a first end, and a micro-diameter tube (MDT) extending from a second end. The MDT is sealed at a distal end to the filling port and at a proximal end to the cavity of the ADID, so as to establish an integral, cell-filling pathway there-within.

### RELATED APPLICATIONS

The present disclosure claims benefit of and priority to U.S. provisional application No. 62/929,619, filed Nov. 1, 2019, and U.S. provisional application No. 62/930,504, filed Nov. 4, 2019, each disclosure of which is herein incorporated by reference it its entirety.

### BACKGROUND

Transporting a bioartificial organ (BAO), or other implantable medical device, especially those of smaller size (e.g., finger-tip size or smaller), can be quite challenging. Given their size, improper handling can lead to damage or sterility-breach of the devices, which, if detected, are discarded, or if undetected may result in potential risk to patients. Moreover, such devices, if required to be filled with a substance, or, multiple steps in a manufacturing process, also requires delicate handling, as well as associated structure to aid in filling and/or manufacturing steps. Improper handling can lead to, yet again, potential risk to patients. This is particularly problematic in semi-automated or automated manufacture of a BAO for treatment of retinal disorders.

### SUMMARY OF SOME OF THE EMBODIMENTS

In some embodiments of the present disclosure, a medical device system is provided and includes a medical device, and/or, in some embodiments, an analyte diffusive implantable device (ADID) having a cavity, a cell-scaffolding material (CSM) arranged within the cavity and configured to hold or otherwise retain a plurality of living cells, and a porous membrane at least partially enclosing the cavity, wherein at least a portion of the membrane is adhered to the CSM by a high durometer adhesive which creates a hemispheric seal at both ends of the membrane. The system also includes a filling port having docking means at a first end, and a micro-diameter tube (MDT) extending from a second end. The MDT is sealed at a distal end to the filling port and at a proximal end to the cavity of the ADID, so as to establish an integral, cell-filling pathway there-within.

In such embodiments, at least one of the following additional features, functionality, structure, steps, and/or clarifications (and in some embodiments, a plurality of, and in some embodiments, all of) can be included, leading to yet further embodiments:
the filling port corresponds to (or includes, comprises) a temporary filling port;
at least one anchor arranged on at least one end of the device;
   the anchor is configured to affix the device to tissue;
   the anchor is configured to be held by a clip, where the clip can be configured to enable packaging and/or transfer of the device;
and
a clip, which can include at least one of (or a plurality of, or all of):
   a first jaw having a corresponding first distal jaw end;
   a second jaw having a corresponding second distal jaw end configured for movement relative to the first jaw end;
   a first jaw extension integral with the first jaw;
   a second jaw extension integral with the second jaw; and/or
   at least two proximal extensions integral with the first jaw;
   the first extension can include any or all of a first projection, a first receiving area and a second receiving area;
   the second extension can include a flexible projection;
   the first receiving area can be configured to receive an end of the flexible projection;
   the second receiving area can be configured to receive the second jaw extension and move therein; and
   the first distal end of the first jaw can be closed with respect to the second distal end of the second jaw in a resting position.

In some embodiments, an analyte diffusive system is provided and includes a medical device, and/or, in some embodiments an analyte diffusive implantable device (ADID) having a cavity, a cell-scaffolding material (CSM) arranged within the cavity and configured to hold or otherwise retain a plurality of living cells, a porous membrane at least partially enclosing the cavity, wherein at least a portion of the membrane is adhered to the CSM by a high durometer adhesive which creates a hemispheric seal at both ends of the membrane, and a filling port configured for connection to an end of the device, wherein the port is configured to be in communication with the cavity and sealed thereto, to provide access to the cavity to enable cells to be received in the cavity, and as a polymer molded structure so as to allow an injection system to seal to thereto.

In such embodiments, at least one of the following additional features, functionality, structure, steps, and/or clarifications (and in some embodiments, a plurality of, and in some embodiments, all of) can be included, leading to yet further embodiments:
a micro-diameter tube (which can also be, in some embodiments, a “tube” of larger size) having an inner micron-sized lumen (for example) configured to flow cells from an injection system into the cavity;
   a first end of the tube is configured to connect to an end of the implantable device,
   a second end of the tube is configured to connect to the port assembly, and
   cells are delivered from the injection system into the cavity from the port assembly and through the tube;
the tube can include a wall thickness and/or material configured to maintain a diameter geometry for delivery of cells through the tube and maintain the diameter geometry at a fill pressure of 0-150 psi (for example);
the tube is sealed to the port assembly with a first adhesive;
the tube is sealed within the CSM and membrane with a second adhesive, having properties distinct from properties associated with a first adhesive;
the tube is configured for preferential release from a first adhesive;
a force required to preferentially release the sealed filling port from within the device requires between 1-50 Newtons (for example);
filling of cells within the cavity is selected from the group consisting of: capillary action, positive displacement, vacuum, servo driven, peristaltic action, and combinations thereof;
a clip;
   the clip is configured to enable packaging and/or transfer of the device, which can include at least one of (and in some embodiments a plurality of, and in some embodiments all of):
      a proximal end and a distal end;
      a first jaw having a corresponding first distal jaw end;
      a second jaw having a corresponding second distal jaw end configured for movement relative to the first jaw end;
      a first jaw extension integral with the first jaw;
      a second jaw extension integral with the second jaw; and/or
      at least two proximal extensions integral with the first jaw;
   the first extension includes a first projection, a first receiving area and a second receiving area;
   the second extension includes a flexible projection;
   the first receiving area being configured to receive an end of the flexible projection;
   the second receiving area being configured to receive the second jaw extension and move therein; and
   the first distal end of the first jaw is closed with respect to the second distal end of the second jaw in a resting position.

In such embodiments, a cell transferring method for transferring cells into an analyte diffusive device is provided and includes providing the analyte diffusive system according to any of the disclosed embodiments, sealing the tube with the device and the port assembly, transferring cells into the device via the port assembly and tube using the injection system, wherein the transferred cells are stored on the CSM within cavity, and removing at least one of the port assembly and tube from the device.

In some embodiments, an aseptic transferring method for transferring or otherwise moving an implantable device is provided and includes providing an analyte diffusive implantable device (ADID), providing a clip, holding the device with the clip, wherein the clip attaches to the device either at one end of the device or via an anchor attached to the end of the device, and moving the device via the clip through automated manufacturing unit operations to fill the device.

In some embodiments, an aseptic method to maintain orientation of device during storage and use for surgical implant is provided and includes providing an analyte diffusive system including an analyte diffusive implantable device (ADID), providing a clip, holding the ADID with the clip, where the clip attaches to the ADID either at one end of the ADID or via an anchor attached to the end of the ADID and orients the ADID vertically and centrally in a packaging system containing cell nutrient media, and optionally aseptic transferring of the ADID from the packing system to a sclerotomy incision and facilitating positioning of the ADID and release of the ADID into the incision and vitreous cavity.

These and other embodiments, objects and advantages will become even more evident with reference to the concurrently filed figures, a brief description of which is set out below, and following detailed description of at least some of the embodiments.

### BRIEF DESCRIPTION OF THE FIGURES

FIG. 1 illustrates an assembled view of a retaining system for filling, holding, and/or retaining an biomedical implant, according to some embodiments of the disclosure.

FIG. 2 illustrates an enlarged, side view of an implant and clip for the system of FIG. 1, according to some embodiments of the disclosure.

FIG. 3A illustrates an enlarged, side, cross-sectional view of an implant and anchor of the assembly, according to some embodiments of the disclosure.

FIG. 3B illustrates an assembled view of a retaining system for filling, holding, and/or retaining a biomedical implant, according to some embodiments of the disclosure.

FIG. 4 illustrates a perspective view of an anchor for the implant, for the assembly, according to some embodiments of the disclosure.

FIG. 5A illustrates a side view of a clip of the assembly, according to some embodiments of the disclosure.

FIG. 5B illustrates a perspective view of the clip of FIG. 5A, according to some embodiments of the disclosure.

### DETAILED DESCRIPTION OF AT LEAST SOME OF THE EMBODIMENTS

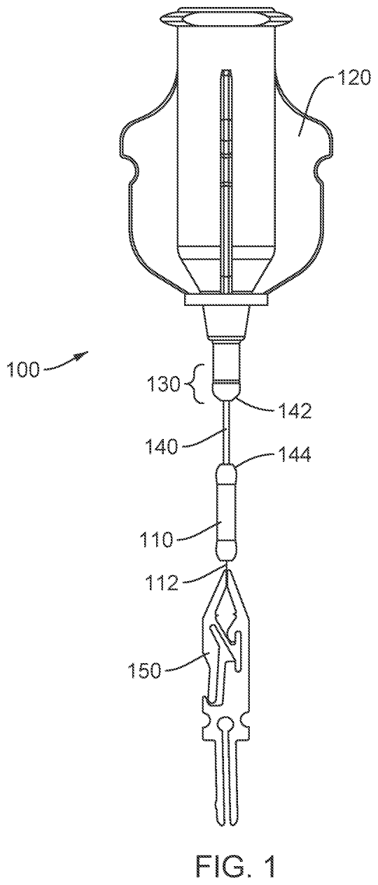

FIGS. 1-5B illustrate embodiments of the present disclosure. FIG. 1 illustrates a first-side view of a medical device system, according to some embodiments, and includes an analyte diffusive implantable device (ADID) 110, which includes a cavity, and a cell-scaffolding material (CSM) arranged within the cavity and configured to hold or otherwise retain a plurality of living cells engineered to secrete a therapeutic protein-based drug (according to some embodiments). The system can also include a porous hollow fiber membrane at least partially enclosing the cavity (see U.S. Pat. Nos. 7,115,257, 6,361,771, 9,265,814, 10,195,140, each of which is herein incorporated by reference in its entirety). At least a portion of the membrane is adhered to the CSM by a high durometer adhesive (for example, with respect to some embodiments) which can create a preferably hemispheric seal at one, and preferably, both ends of the membrane. Also included is a filling port 120 (which in some embodiments can be referred to as a filling port assembly), having a docking means 130, which may also be referred to a port assembly, at a first end, and a micro-diameter tube (MDT) 140 extending from a second end. The MDT is preferably sealed (and preferably, hermetically) at a distal end 142 to the filling port 120, and at a proximal end 144 to the cavity of the ADID 110, so as to establish an integral, cell-filling pathway there-within.

In some embodiments, the ADID/system 110 includes at least one anchor 112 arranged on at least one end of the device, where the anchor can be configured to affix the device 110 to tissue (for example). The anchor 112 can also be configured to at least one of be received and held by a clip-device 150 (“clip”). The clip 150 can be configured to enable packaging and/or transfer of the ADID.

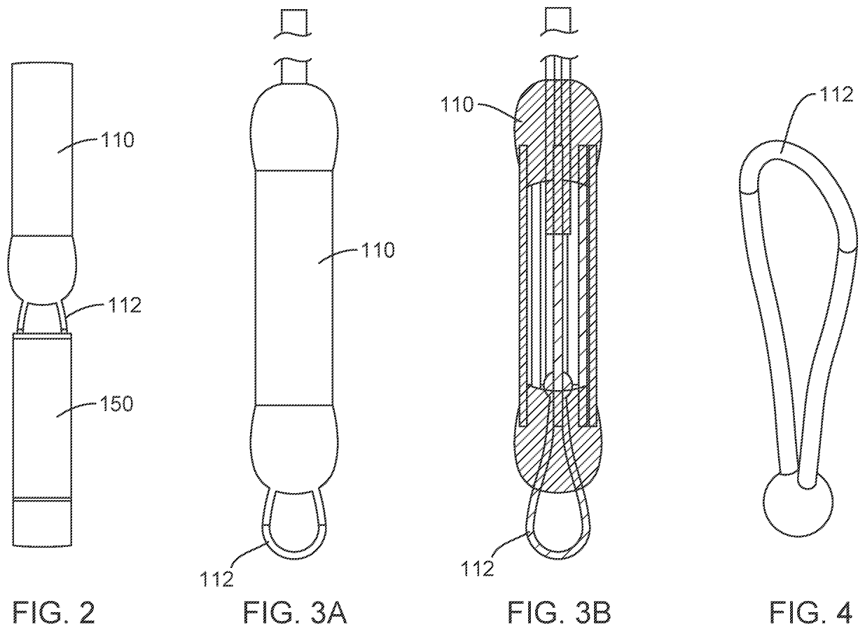

In such embodiments, the filling port 120 corresponds to (or includes, comprises) a temporary filling port, such that, it is only used to fill the ADID, and then is of no further use and can be removed/discarded/reused. The at least one anchor (in some embodiments), can be a plurality of anchor, and arranged on at least one end of the device is configured to affix the device to tissue. FIGS. 2-4 illustrate various views of the at least one anchor and ADID according to some embodiments. Specifically, FIG. 2 is a second side view of an enlargement of the clip-device 150, the at least one anchor, and the ADID in combination attachment, where the at least one anchor is configured to be held by the clip-device, so as to, in some embodiments, enable packaging and/or transfer of the device.

As shown in FIGS. 3A-B and 4, the at least one anchor can be a loop, which can include a metal or polymer (or a combination thereof), and multiple anchors of this configuration can be used to couple with the ADID 110 (and one or more of which used to anchor the ADID to tissue. In some embodiments, a single leg of the loop configuration can be considered the at least one anchor). In some embodiments, the at least one anchor includes the loop configuration (as shown in FIGS. 2A-2D), which can include a bulbous end or bulb 112a, which is coupled to the ADID 110 to an internal structure 110a therein (see cross-section, FIG. 3B).

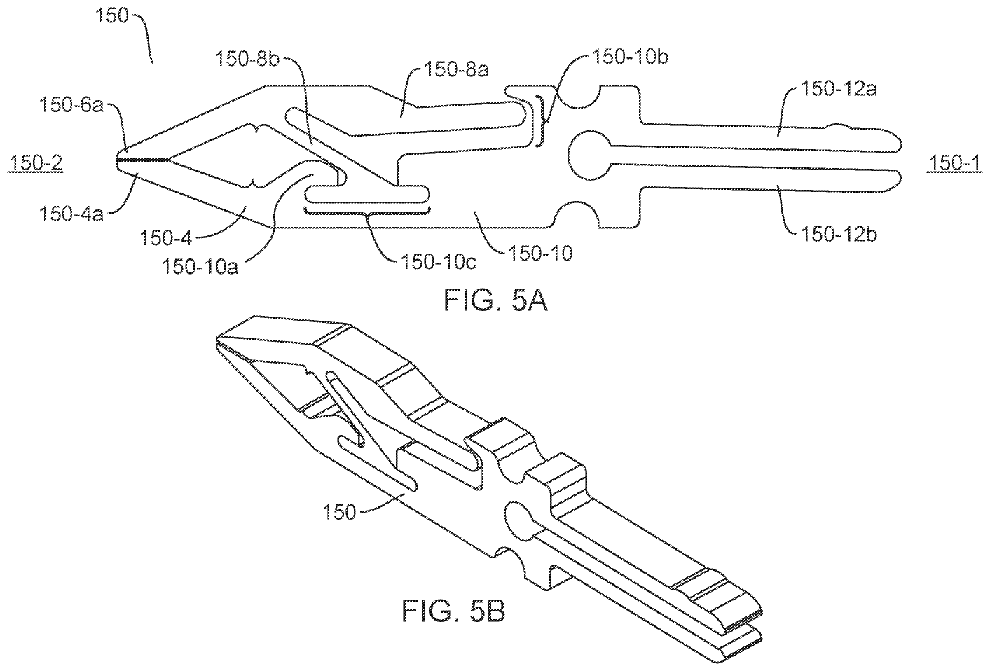

As shown in FIGS. 5A-B, the at least one anchor can be configured to be held by the clip 150, where the clip can include a proximal end 150-1 and a distal end 150-2, a first jaw 150-4 having a corresponding first distal jaw end 150-4a, a second jaw 150-6 having a corresponding second distal jaw end 150-6a configured for movement relative to the first jaw end. A first jaw extension 150-10 may also be included, which can be integral with the first jaw 150-4, a second jaw extension 150-8 integral with the second jaw 150-6, and at least two proximal extensions 150-12a, 150-12b integral with the first jaw 150-4.

In some embodiments, in such this configuration, the first jaw extension 150-10 can include a first projection 150-10a, a first receiving area 150-10b, and a second receiving area 150-10c, the second extension 150-8 can include a flexible projection 150-8a, such that the first receiving area 150-10b can be configured to receive an end of the flexible projection 150-8a, the second receiving area 150-8c can be configured to receive the second jaw extension 150-8b and move therein, and/or the first distal end 150-4a of the first jaw 150-4 can be closed with respect to the second distal end 150-6a of the second jaw 150-6 in a resting position (for example).

In some embodiments, an analyte diffusive system is provided, and includes an analyte diffusive implantable device (ADID) which includes a cavity, a cell-scaffolding material (CSM) arranged within the cavity and configured to hold or otherwise retain a plurality of living cells, and a porous membrane at least partially enclosing the cavity, wherein at least a portion of the membrane is adhered to the CSM by a high durometer adhesive which creates a hemispheric seal at both ends of the membrane, and a filling port configured for connection to an end of the device. The port is configured to be in communication with the cavity and sealed thereto, to provide access to the cavity to enable cells to be received in the cavity, and as a polymer molded structure so as to allow an injection system to seal to thereto.

In such embodiments the micro-diameter tube 140 includes an inner micron-sized lumen configured to flow cells from an injection system into the cavity. Specifically, in some embodiments, the inner diameter of the associated lumen is between about 50-500 μm, and in some embodiments preferably between 100-150 μm. The tube includes a wall thickness and/or material configured to maintain a diameter geometry for delivery of cells through the tube and maintain the diameter geometry at a fill pressure of 0-150 psi (for example), and the tube 140 can be sealed to the port assembly with a first adhesive (or other means familiar to one of skill in the art). The tube 140 can be sealed within the CSM and membrane with a second adhesive (for example), having properties distinct from properties associated with the first adhesive (according to some embodiments).

A first end 140-2 of the tube 140 can be configured to connect to an end 110-2 of the ADID 110, a second end 140-4 of the tube is configured to connect to the port assembly 130, and cells are delivered from the injection system into the cavity of the ADID from the port assembly and through the tube 140. In some embodiments, the tube 140 is configured for preferential release from the first adhesive (e.g., holding the tube 140 to the port assembly 130), in which, a force required to preferentially release the sealed tube 140 from within the device, which in some embodiments, can require between 1-100 Newton-meters, or, in some embodiments, preferably between 30-35 Newton-meters.

The filling of cells within the cavity of the ADID 110 can be any one or more of: capillary action, positive displacement, vacuum, servo driven, peristaltic action, and combinations thereof.

In some embodiments, a cell transferring method for transferring cells into an analyte diffusive device is provided, or any device medical or otherwise (for that matter), and can include, in some embodiments for example, providing an analyte diffusive system according to any of the disclosed embodiments (e.g., those noted above), and sealing the tube with the ADID device and the port assembly 130. The method can further include, for example, transferring cells into the device via the port assembly 130 and tube 140 using, for example, an injection system. In some embodiments, the transferred cells are stored on/within the CSM within a cavity of the ADID 110, and removing at least one of the port assembly 130 and tube from the ADID 110.

Still further in some embodiments, an aseptic transferring method for transferring or otherwise moving an implantable device (or any device medical or otherwise, for that matter) is provided, the method includes providing an analyte diffusive implantable device (ADID), providing a clip, holding the device with the clip, where the clip attaches to the device either at one end of the device or via an anchor attached to the end of the device, and moving the device via the clip through automated manufacturing unit operations to fill the device.

In some embodiments, an aseptic method to maintain orientation of a device during storage and/or use for a surgical implant (or any device medical or otherwise, for that matter), the method includes providing an analyte diffusive system (or any device, medical or otherwise for that matter) including an analyte diffusive implantable device (ADID), providing a clip, holding the ADID with the clip, wherein the clip attaches to the ADID either at one end of the ADID or via an anchor attached to the end of the ADID and orients the ADID vertically and centrally in a packaging system containing cell nutrient media, and optionally aseptic transferring of the ADID from the packing system to a sclerotomy incision and facilitating positioning of the ADID and release of the ADID into the incision and vitreous cavity.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims, equivalents thereto, and any claims supported by the present disclosure, inventive embodiments may be practiced otherwise than as specifically described and claimed. Inventive embodiments of the present disclosure are directed to each individual feature, system, article, material, kit, method, and step, described herein. In addition, any combination of two or more such features, systems, articles, materials, kits, methods, and steps, if such features, systems, articles, materials, kits, methods, and steps, are not mutually inconsistent, is included within the inventive scope of the present disclosure. Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, various inventive concepts may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is currently claimed:

1. A medical device system comprising:
an analyte diffusive implantable device (ADID) which comprises:
a cavity;
a cell-scaffolding material (CSM) arranged within the cavity and configured to hold or otherwise retain a plurality of living cells;
and
a porous membrane at least partially enclosing the cavity, wherein at least a portion of the membrane is adhered to the CSM by a high durometer adhesive which creates a hemispheric seal at both ends of the membrane;
and
a filling port having:
docking means at a first end, and
a micro-diameter tube (MDT) extending from a second end,
wherein the MDT is sealed at a distal end to the filling port and at a proximal end to the cavity of the ADID, so as to establish an integral, cell-filling pathway there-within.

2. The system of claim 1, wherein the filling port comprises a temporary filling port.

3. The system of claim 1 or 2, further comprising at least one anchor arranged on at least one end of the device.

4. The system of claim 3, wherein the anchor is configured to affix the device to tissue.

5. The system of any of claims 3-4, wherein the anchor is configured to be held by a clip.

6. The system of claim 5, wherein the clip is configured to enable packaging and/or transfer of the device.

7. The system of claim 5 or 6, wherein the clip comprises:
a proximal end and a distal end;
a first jaw having a corresponding first distal jaw end;
a second jaw having a corresponding second distal jaw end configured for movement relative to the first jaw end;
a first jaw extension integral with the first jaw;
a second jaw extension integral with the second jaw; and
at least two proximal extensions integral with the first jaw;
wherein:
the first extension includes a first projection, a first receiving area and a second receiving area;
the second extension includes a flexible projection;
the first receiving area being configured to receive an end of the flexible projection;
the second receiving area being configured to receive the second jaw extension and move therein; and
the first distal end of the first jaw is closed with respect to the second distal end of the second jaw in a resting position.

8. An analyte diffusive system comprising:
an analyte diffusive implantable device (ADID) which comprises:
a cavity;
a cell-scaffolding material (CSM) arranged within the cavity and configured to hold or otherwise retain a plurality of living cells;
and
a porous membrane at least partially enclosing the cavity, wherein at least a portion of the membrane is adhered to the CSM by a high durometer adhesive which creates a hemispheric seal at both ends of the membrane;
and
a filling port configured for connection to an end of the device, wherein the port is configured:
to be in communication with the cavity and sealed thereto,
to provide access to the cavity to enable cells to be received in the cavity, and
as a polymer molded structure so as to allow an injection system to seal to thereto.

9. The system of claim 8, further comprising a micro-diameter tube having an inner micron-sized lumen configured to flow cells from an injection system into the cavity.

10. The system of claim 9, wherein:
a first end of the tube is configured to connect to an end of the implantable device,
a second end of the tube is configured to connect to the port assembly, and
cells are delivered from the injection system into the cavity from the port assembly and through the tube.

11. The system of claim 9 or 10, wherein the tube includes a wall thickness and/or material configured to maintain a diameter geometry for delivery of cells through the tube and maintain the diameter geometry at a fill pressure of 0-150 psi.

12. The system of any of claims 9-11, wherein the tube is sealed to the port assembly with a first adhesive.

13. The system of any of claims 9-11, wherein the tube is sealed within the CSM and membrane with a second adhesive, having properties distinct from properties associated with the first adhesive of claim 12.

14. The system of any of claims 9-13, wherein the tube is configured for preferential release from the first adhesive comprising the device as described in claim 13.

15. The system of claims 9-14, wherein a force required to preferentially release the sealed filling port from within the device requires between 1-50 Newtons.

16. The system of any of claims 8-15, wherein filling of cells within the cavity is selected from the group consisting of: capillary action, positive displacement, vacuum, servo driven, peristaltic action, and combinations thereof.

17. The system of any of claims 8-16, further comprising a clip.

18. The system of claim 17, wherein the clip is configured to enable packaging and/or transfer of the device.

19. The system of claim 17 or 18, wherein the clip comprises:
a proximal end and a distal end;
a first jaw having a corresponding first distal jaw end;
a second jaw having a corresponding second distal jaw end configured for movement relative to the first jaw end;
a first jaw extension integral with the first jaw;
a second jaw extension integral with the second jaw;
and
at least two proximal extensions integral with the first jaw;
wherein:
the first extension includes a first projection, a first receiving area and a second receiving area;
the second extension includes a flexible projection;
the first receiving area being configured to receive an end of the flexible projection;
the second receiving area being configured to receive the second jaw extension and move therein; and
the first distal end of the first jaw is closed with respect to the second distal end of the second jaw in a resting position.

20. A cell transferring method for transferring cells into an analyte diffusive device or system, the method comprising:
providing the analyte diffusive system of any of claims 1-19;
sealing the tube with the device and the port assembly;
transferring cells into the device via the port assembly and tube using the injection system, wherein the transferred cells are stored on the CSM within cavity;
and
removing at least one of the port assembly and tube from the device.

21. An aseptic transferring method for transferring or otherwise moving an implantable device or system, the method comprising:
providing an analyte diffusive implantable device (ADID)
providing a clip;
holding the device with the clip, wherein the clip attaches to the device either at one end of the device or via an anchor attached to the end of the device;
and
moving the device via the clip through automated manufacturing unit operations to fill the device

22. An aseptic method to maintain orientation of a device or system during storage and use for a surgical implant, the method comprising
providing an analyte diffusive system including an analyte diffusive implantable device (ADID);
providing a clip;
holding the ADID with the clip, wherein the clip attaches to the ADID either at one end of the ADID or via an anchor attached to the end of the ADID and orients the ADID vertically and centrally in a packaging system containing cell nutrient media;
and
optionally aseptic transferring of the ADID from the packing system to a sclerotomy incision and facilitating positioning of the ADID and release of the ADID into the incision and vitreous cavity.

23. A system, apparatus, device or method according to any disclosed embodiment.
